(19) United States
(12) Patent Application Publication
(10) Pub. No.: US 2021/0040146 A1
(43) Pub. Date: Feb. 11, 2021

(54) PROTEIN PURIFICATION PROCESS

(71) Applicant: EXOTHERA S.A., Nivelles (BE)

(72) Inventors: Sushobhan Bandyopadhyay, Nivelles (BE); José Castillo, Brussels (BE); Vasily Medvedev, Nivelles (BE)

(21) Appl. No.: 16/965,792

(22) PCT Filed: Jan. 29, 2019
(86) PCT No.: PCT/EP2019/052104

§ 371 Date: Jul. 29, 2020

Related U.S. Application Data
(60) Provisional application No. 62/731,385, filed on Sep. 14, 2018.
(60) Provisional application No. 62/623,812, filed on Jan. 30, 2018.

(51) Int. Cl. C07K 1/16; C07K 16/06; C07K 16/24

(52) U.S. Cl. CPC C07K 1/165; C07K 16/241; C07K 16/065

(57) ABSTRACT

The current disclosure describes a method for purifying a protein, wherein said protein is present in a feed, and wherein said method comprises a multimodal chromatography step, wherein said feed is contacted with a multimodal ion exchanger comprising a ligand with a hydrophobic moiety and a charged moiety and wherein binding of said protein of interest to said exchanger occurs under high conductivity conditions.

### TECHNICAL DOMAIN This disclosure concerns a method for protein purification.

### BACKGROUND

With the increasing number of protein therapeutic candidates, especially monoclonal antibodies (mAbs) entering various stages of development, biopharmaceutical companies are increasingly looking at innovative solutions to deliver this pipeline. For antibody manufacturing process development, maintaining desired quality attributes while reducing time to market, maintaining cost effectiveness, and providing manufacturing flexibility are key issues in today's competitive market. Since antibody therapies may require large doses over a long period of time, the drug substance must be produced in large quantities with cost and time efficiency to meet clinical requirements and pave the way toward commercialization. This is also the case for recombinant therapeutic proteins other than monoclonal antibodies including but not limited to fusion proteins, therapeutic enzymes and antibody fragments.

Generally, proteins are produced by cell culture, using cell lines, bacterial cell lines or viruses engineered to produce the protein of interest. The cell lines are fed with a complex growth medium comprising sugars, amino acids, and growth factors. For use as a human therapeutic, the target molecules or target protein expressed by the cultured cells must be treated to reach a high level of purity. After initial purifications, a final purification step, often called polishing, is used to obtain the desired final purity.

Although required for achieving a high level of purity, polishing may lead to significant protein loss. The majority of the polishing methodologies known to date comprise several polishing substeps, again contributing to a loss of desired product.

It is the aim of the present disclosure to provide a method which allows polishing of a protein feed with a minimum of protein loss and assurance of high protein quality. Second, it is also the aim to provide a methodology with a limited amount of operational steps that still provides high yield of protein and with a significant reduction of operation expenses (OPEX).

### SUMMARY

Disclosed herein are methods of using a multimodal ion exchanger comprising hydrophobic moieties and which is loaded under high conductivity conditions. These conditions cause a kosmotropic effect, wherein the protein of interest will be more exposed to the hydrophobic moieties of the exchanger or have a higher affinity to the latter, allowing a hydrophobic interaction between said protein and the exchanger. Allowing the method to run under high conductivity conditions improves the selectivity of the protein of interest towards its monomeric form. In addition, less protein aggregates are formed under these conditions. At the same time all the other major attributes of ion exchange are arguably preserved: reduction of host cell proteins, host cell DNA, and reduction of viral particles. Use of this type of multimodal ion exchanger reduces intermediate steps required for conditioning the feed.

Disclosed herein are methods for purifying a molecule. In some embodiments the molecule is a protein, peptide, amino acid or nucleic acid. In some embodiments the molecule is a protein. In some embodiments, the protein is present in a feed. In some embodiments, the method comprises a multimodal chromatography step, wherein the feed is contacted with a multimodal ion exchanger. In some embodiments, the multimodal ion exchanger comprises a ligand with a hydrophobic moiety and a charged moiety. In some embodiments, the binding of the protein of interest to the exchanger occurs under high conductivity conditions. In some embodiments, the feed is supplemented with an adequate amount of salt or a combination of salts prior to the multimodal chromatography step. In some embodiments, the feed is supplemented with an adequate amount of ammonium sulfate, sodium sulfate, potassium sulfate, ammonium phosphate, sodium phosphate, potassium phosphate, potassium chloride, sodium chloride or a mixture thereof prior to the multimodal chromatography step. In some embodiments, the salt concentration of the feed during binding is between 0.5 M and 3 M. In some embodiments, the salt concentration of the feed during binding is between 1 M and 2 M. In some embodiments, the charged moiety is positively or negatively charged. In some embodiments, the multimodal exchanger has both positively and negatively charged moieties. In some embodiments, the feed is supplemented with an adequate amount of an acidic solution or with an adequate amount of an alkaline solution prior to multimodal chromatography step. In some embodiments, the binding occurs at a pH of about 7 to 9. In some embodiments, the multimodal chromatography step is used as a polishing step. In some embodiments, the multimodal chromatography step is the sole polishing step. In some embodiments, the polishing step is preceded by a clarification step of a cell culture harvest and/or a chromatography step. In some embodiments, the protein is eluted from the multimodal exchanger by gradient elution, by gradually decreasing the pH of an elution buffer below 7 and/or by gradually decreasing the salt concentration in an elution buffer below 0.5 M. In some embodiments, the protein is eluted from the multimodal exchanger by isocratic elution with an elution buffer, wherein the elution buffer has a salt concentration of between 10 mM and 500 mM and/or a pH of between 5.5 and 7. In some embodiments, the feed comprises inactivated viruses. In some embodiments, the feed for multimodal chromatography is a flow-through fraction of a chromatography step or a fraction derived thereof. In some embodiments, the protein is an antibody. In some embodiments, the method is performed in batch mode or continuous chromatography mode.

Disclosed herein are kits. In some embodiments, the kit comprises a multimodal chromatography resin. In some embodiments, the multimodal chromatography resin comprises a ligand with a hydrophobic moiety and a charged moiety. In some embodiments, the kit comprises a buffer with a salt concentration of between 0.5 and 3 M and/or a conductivity of above 75 mS/cm.

Disclosed herein are multimodal ion exchangers. In some embodiments, the multimodal ion exchanger comprises a ligand with a hydrophobic moiety and a charged moiety. In some embodiments the molecule is a protein a protein is bound to the hydrophobic moiety. In some embodiments, the protein prior to loading is present in a buffer with a salt concentration of between 0.5 and 3 M and/or a conductivity of above 75 mS/cm. In some embodiments, the buffer comprises a salt concentration of between 0.5 and 3 M and/or conductivity of above 75 mS/cm. In some embodiments, the buffer comprises a pH of about 7 to 9.

### FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart depicting an overview of large scale commercial process of protein production and purification.

FIG. 2 is a flow chart depicting an overview of the method for purifying a protein according to an embodiment of the disclosure.

FIG. 3 shows a chromatogram accompanying the experiment performed in example 2.

### DEFINITIONS

Unless otherwise defined, all terms used herein, including technical and scientific terms, have the meaning as commonly understood by one of ordinary skill in the art to which this invention belongs. By means of further guidance, term definitions are included to better appreciate the teaching of the present disclosure.

As used herein, the following terms have the following meanings:

“A”, “an”, and “the” as used herein refers to both the singular and plural unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation. The expression “1% w/w” refers to what can be understood as 1g of respective component per 100 g of the formulation, the expression “1% w/v” refers to what could be understood as 1g of respective component per 100 mL of the formulation, the expression “1% v/v” refers to what can be understood as 1 mL of respective component per 100 mL of formulation.

“Cell culture harvest”, “culture harvest” and “harvest” are used as synonyms and refer to the unclarified cell culture obtained from culturing cells in a bioreactor. The cultured cells or the grown cells also are referred to as host cells.

“Bioreactor” refers to any device or system that supports a biologically active environment, for example for cultivation of cells or organisms for production of a biological product. This would include cell stacks, roller bottles, shakes, flasks, stirred tank suspension bioreactors, high cell density fixed bed perfusion bioreactors, etc.

“Purification” refers to the substantial reduction of the concentration of one or more target impurities or contaminants relative to the concentration of the molecule of interest, such as a protein of interest.

“Protein” refers to any of a class of nitrogenous organic compounds which have large molecules composed of one or more chains of amino acids. “Protein” may be any sort of protein such as (monoclonal) antibodies, antibody fragments, fusion proteins, enzymes, recombinant proteins, peptides, polypeptides or other biomolecules expressed by cells.

“Antibody” refers to any immunoglobulin molecule, antigen-binding immunoglobulin fragment or immunoglobulin fusion protein, monoclonal or polyclonal, derived from human or other animal cell lines, including natural or genetically modified forms such as humanized, human, chimeric, synthetic, recombinant, hybrid, mutated, grafted, and in vitro generated antibodies. Commonly known natural immunoglobulin antibodies include IgA (dimeric), IgG, IgE, IgG and IgM (pentameric).

An “antibody charge variant” as used herein is an antibody or fragment thereof wherein the antibody or fragment thereof has been modified from its native state such that the charge of the antibody or fragment thereof is altered. Antibody charge variants are often referred to as acidic, neutral and/or basic antibody species.

“Protein aggregate” or “aggregate of a protein” refers to an association of at least two protein molecules. The association may be either covalent or non-covalent without respect to the mechanism by which the protein molecules are associated. The association may be direct between the protein molecules or indirect through other molecules that link the protein molecules together. Examples of the latter include but are not limited to disulfide linkages with other proteins, hydrophobic associations with lipids, charge associations with DNA, affinity associations with leached protein A, or mixed mode associations with multiple components.

“Viral inactivate” means that a feed comprising a protein of interest further comprises inactivated viruses or viral particles previously existing in their active form in a bioreactor.

With “flow-through fraction” is meant here at least part of the loaded protein-containing fraction which leaves the chromatographic column at substantially the same velocity as the elution fluid. This fraction is substantially not retained on the column during elution. Hence the conditions are chosen such that not the antibodies but the impurities are bound to the respective chromatographic materials.

“Flocculation” refers to the aggregation, precipitation and/or agglomeration of soluble (e.g., chromatin components) and/or insoluble (e.g., cells) particles caused by the addition of a suitable flocculating agent to a suspension. By increasing the particle size of the insoluble components present in the suspension, the efficiency of solid/liquid separations, such as by filtration or centrifugation, is improved. Flocculation of a cell culture leads to the formation of “floccules” which comprise host cell impurities such as cell material including cells and cell debris or host cell proteins, DNA or other components present therein.

As used herein, “polishing” refers to a step, preferably a chromatographic step, used to remove residual host cell impurities, product-related impurities (product fragments and/or aggregated species or charge variants) and virus contaminants, from a feed thereby (further) purifying the protein of interest. Polishing is often preceded by a protein capture step.

“Mixed-mode” and “multimodal” chromatography are used interchangeably herein and refer to a chromatographic method wherein separation of components in a mobile phase is based on more than one interaction type between a stationary phase and the components of the mobile phase. Multimode chromatography is, for example, based on simultaneous hydrophobic or non-hydrophobic interactions and electrostatic interactions between the mobile phase and the stationary phase.

A “multimodal ion exchanger” refers to a component of the solid phase which is suited for use in multimodal chromatography and which has a charged moiety. The terms “multimodal ion exchanger”, “mixed-mode ion exchanger”, “multimodal ion exchange ligand” and “mixed-mode ion exchange ligand” are used herein as synonyms.

As used herein the terms “kosmotropic” or “kosmotropes” refer broadly to substances that, without being bound by theory are thought to contribute to the stability and structure of water-water interactions. Kosmotropes typically cause water molecules to favorably interact, which also stabilizes intermolecular interactions in macromolecules. Kosmotropes can be ionic and/or nonionic.

“High Conductive Conditions” are to be understood as those conditions or measures that exist during loading of a feed or buffer of such feed and binding of a feed or buffer of such feed to a resin whereby the conductivity of the feed or buffer of said feed is above 75 mS/cm, more preferably above 85 mS/cm. Often, high conductivity is obtained by having an increased salt concentration of the feed buffer, e.g. above 1 M NaCl and more. The skilled person is aware that a different salt will have a different effect on conductivity.

The term “solid phase” is used to mean any non-aqueous matrix to which one or more ligands can adhere or alternatively, in the case of size exclusion chromatography, it can refer to the gel structure of a resin. The solid phase can be any matrix capable of adhering ligands in this manner, e.g., a purification column, a discontinuous phase of discrete particles, a membrane, filter, gel, etc. Examples of materials that can be used to form the solid phase include polysaccharides (such as agarose and cellulose) and other mechanically stable matrices such as silica (e.g. controlled pore glass), poly(styrenedivinyl)benzene, polyacrylamide, ceramic particles and derivatives of any of these.

“Buffering compound” refers to a chemical compound employed for the purpose of stabilizing the pH of an aqueous solution within a specified range. Phosphate is one example of a buffering compound. Other common examples include but are not limited to compounds such as acetate, citrate, borate, MES, Tris, and HEPES, phosphate buffered saline (PBS) among many others.

“Buffer” refers to an aqueous formulation comprising a buffering compound and other components required to establish a specified set of conditions to mediate control of a chromatography method. The term “equilibration buffer” refers to a buffer formulated to create the initial operating conditions. “Wash buffer” refers to a buffer formulated to displace unbound contaminants from a chromatography support. “Elution buffer” refers to a buffer formulated to displace the one or more components from the chromatography matrix.

An “adequate amount” of a solution is used to describe a solution that, when mixed with the feed or with the chromatography resin will promote binding or adsorption of the majority of the protein of interest to the resin, but it will not promote substantial binding of impurities to that resin. For each purification process the optimum pH, the preferred type of salt system and the optimum amounts in the adjusting solutions have to be established.

### DETAILED DESCRIPTION

In a first aspect, a method for purifying a protein is disclosed, wherein said protein is present in a feed, and wherein said method comprises a multimodal chromatography step, wherein said feed is contacted with a multimodal ion exchanger comprising a ligand with a hydrophobic moiety and with a charged moiety, thereby allowing binding of the protein to said exchanger, and wherein said binding occurs under high conductivity conditions.

Multimodal or mixed-mode chromatography combines different types of interactions such as ionic interaction, hydrogen bonding and hydrophobic interaction to allow the separation of components in a mixture (i.e. a feed comprising the protein of interest). The separation properties of the ligand are modulated by adjusting the conditions wherein interaction between the components of the feed and the ligand takes place. In the currently disclosed method, the driving forces for binding and elution of the protein of interest to the multimodal ligand are specifically adapted by adjusting the chromatography conditions in order to improve the chromatographic separation of the components such that it can be performed more accurately and more selectively towards a specific protein of interest and even towards specific forms of the protein of interest (e.g. the monomeric form of an antibody).

According to the method for protein purification disclosed herein, the multimodal chromatography step used is based on a ligand with a hydrophobic moiety as well as a charged moiety and accordingly, has both ion exchange as well as hydrophobic interaction capacities. Said charged moiety may either be a positively charged moiety (anionic) or a negatively charged moiety (cationic).

Various mixed mode chromatography ligands are available commercially. Ligands well known in the art and which are compatible with the methodology as currently described are Capto-Adhere™ or Capto-Adhere™ ImpRes, Capto MMC HiScreen, Nuvia™ aPrime. In one embodiment the multimodal ion exchanger comprises a negatively charged group.

In an embodiment the multimodal ion exchanger comprises a positively charged group (preferably an amine or a quaternary ammonium ion) and an aromatic ring structure.

These functionalities can be accommodated either in separate substituents/ligands as a more or less stochastic mixture of ligands or with both functionalities present in the same substituent/ligand.

Particularly useful are ligands defined by the formula

R₁—R₂—N(R₃)—R₄—R₅

wherein
R₁ is a substituted or non-substituted phenyl group;
R₂ is a hydrocarbon chain comprising 0-4 carbon atoms;
R₃ is a hydrocarbon chain comprising 1-3 carbon atoms
R₄ is a hydrocarbon chain comprising 1-5 carbon atoms and
R₅ is OH or H.

Examples of such ligands include but are not limited to N-Benzyl-N-methyl ethanol amine or N,N-dimethyl benzylamine. In another or further embodiment, said ligand of the multimodal resin further comprises hydrophobic and non-hydrophobic moieties such as a hydrogen moiety, which may allow further interaction with the protein of interest, depending on the binding conditions used.

In an embodiment, said multimodal exchanger has both positively and negatively charged moieties.

In an embodiment, said multimodal exchanger comprise additional moieties, allowing additional interaction functionalities other than ion exchange of said column, such as hydrophobic-interaction-enabling moieties or hydrogen-bonding-enabling moieties or any other known in the art.

Without wishing to be bound to any theory, the high conductivity conditions as used in the current disclosure were found to result in a kosmotropic effect, wherein the protein of interest is more exposed to the hydrophobic moieties of the exchanger, hence allowing a hydrophobic interaction between said protein and the exchanger. Thus, by loading and binding the protein feed onto the multimodal ion exchanger under high conductivity conditions, a shift towards the hydrophobic properties of the multimodal exchanger is obtained, whereby interaction of the protein of interest with the multimodal ion exchanger is based on the hydrophobic properties of said protein and the hydrophobic moiety of the multimodal ion exchange ligand.

It was found that the above mentioned shift in chromatography mode allows for successful modulation of the separation properties of the multimode chromatography resin, e.g. selectivity towards the monomeric form of a protein, e.g. antibody. In addition, the shift in chromatography mode also allows to selectively separate antibody charge variants from one another. These antibody charge variants are often referred to as the acidic, the neutral or the basic antibody species. Simultaneously, all major attributes of ion exchange chromatography such as reduction of host cell proteins and DNA, as well as reduction in viral particles are preserved as well. Hence, the method allows obtaining a highly pure protein sample, of a high quality.

In one embodiment, the multimodal chromatography step comprises a classical packed bed column containing a resin, a column containing monolith material, a radial column containing suitable chromatographic medium, an adsorption membrane unit, or any other multimodal chromatography device known in the art with the appropriate medium and the ligands as disclosed. In the chromatographic column the chromatographic material may be present as particulate support material to which the ligands according to the disclosure are attached. The current disclosure is provided with respect to a chromatography step comprising a column containing a resin, however, it is clear to the skilled artisan that the teachings of the disclosure can equally be applied with respect to a chromatography step comprising a column containing an adsorption membrane.

The multimodal chromatography resin may be practiced in a packed bed column, a fluidized/expanded bed column, and/or a batch operation where the multimodal resin is mixed with the protein feed for a certain time. A solid phase chromatography support can be a porous particle, nonporous particle, membrane, or monolith. In some embodiments, the solid phase support comprising the multimodal ligand is packed in a column of at least 5 mm internal diameter and a height of at least 25 mm. Such embodiments are useful, e.g., for evaluating the effects of various conditions on a particular protein (e.g. antibody). Another embodiment employs the solid phase support comprising the multimodal ligand, packed in a column of any dimension required to support applications and further scaled-up operations. Column diameter may range from less than 1 cm to more than 1 meter, and column height may range from less than 1 cm to more than 30 cm depending on the requirements of a particular application. Commercial scale applications will typically have a column diameter (ID) of 20 cm or more and a height of at least 20 cm. Appropriate column dimensions can be determined by the skilled artisan.

In some embodiments, the membrane-type chromatography column comprises a support material in the form of one or more sheets to which a ligand with a hydrophobic moiety and a charged moiety (either positive or negative) is attached. The support material may be composed of organic material or inorganic material or a mixture of organic and inorganic material. Suitable organic materials are agarose based materials and methacrylate. Suitable inorganic materials are silica, ceramics and metals.

Conditions of high conductivity, according to the present disclosure refer to conditions where the conductivity is above 75 mS/cm, more preferably above 85 mS/cm, even more preferably between 85 and 120 mS/cm. High conductivity is often obtained by having an increased salt concentration, e.g. above 1 M NaCl and more. The anion of the salt may preferably be selected from the group consisting of phosphate, sulfate, chloride, acetate, bromide, nitrate, chlorate, iodide and thiocyanate ions. The cation of the salt may preferably be selected from the group consisting of ammonium, potassium, sodium, rubidium, lithium, magnesium, calcium and barium ions. Preferred salts are ammonium sulfate, sodium sulfate, potassium sulfate, ammonium phosphate, sodium phosphate, potassium phosphate, potassium chloride and sodium chloride or a mixture thereof. The skilled person is aware that a different salt will affect the conductivity to a different degree. In a preferred embodiment, the salt is selected from the group comprising sodium chloride, ammonium sulfate and potassium phosphate. These groups of salts are especially well suited for the currently disclosed method as they generate ions which strongly promote hydrophobic interactions. Most chromatography devices are adapted with equipment to monitor the conductivity of the mobile phase or buffers used during chromatography, as well as the salt concentration, and/or the temperature thereof. Loading of the feed comprising the protein of interest under high conductivity will accordingly also result in binding of the protein of interest to the resin under high conductivity conditions or high salt concentration.

In an embodiment, loading of the feed and binding of the protein of interest onto the multimodal chromatography resin occurs at a pH of about 7 to 9 and a salt concentration of between 0.5 M to 3 M or a salt concentration corresponding to a conductivity of at least 75 mS/cm, preferably at least 80 mS/cm, preferably at least 85 mS/cm, preferably between 80 and 120 mS/cm. In a further embodiment, said salt concentration is between 1 M and 2 M NaCl or 0.5 M and 1 M potassium phosphate, respectively, or a salt concentration corresponding to a conductivity of above 75mS/cm, more preferably above 85 mS/cm, preferably between 80 and 120 mS/cm. It was observed that binding below 1M NaCl may be too low to shift the interaction mode from anion exchange (AEX) to hydrophobic interaction chromatography (HIC), whereas, above 2M NaCl, a salting out effect can be observed and buffers will become less cost effective. A pH lower than 7 may result in limited binding to the resin, whereas a pH above 9 results in protein deamidation which is an undesired modification of the protein of interest. A typical condition could be an eluate of step (b) or an eluate derived thereof at a 1M to 2M NaCl concentration in a (20 to 100 mM) HEPES or Tris buffer of pH 7 to 8.

In some embodiments, the conditions of the chromatography mobile phase (i.e. the feed) as well as the solid phase (i.e. the chromatography resin) may be adjusted by conditioning or equilibration towards the conditions as described above, prior to performing the multimodal chromatography step. In another embodiment, the conditions of the chromatography mobile phase (i.e. the feed) as well as the solid phase (i.e. the chromatography resin) may already comply with the required conditions, hence omitting the necessity for conditioning or equilibration. Preferably, the conditions used in upstream processing steps which lead to the feed prior to the multimodal chromatography step of the method currently disclosed, are selected in order to be compatible with the multimodal chromatography step, thereby making a substep comprising conditioning of the feed before purifying the protein of interest unnecessary thus saving time and resources when the method is performed as part of a protein production process.

It is to be understood that adjusting or conditioning of the feed might be achieved by any method known to the person skilled in the art including dilution, buffer exchange, titration or any combination thereof.

A conditioning aiming at decreasing the conductivity of a feed could be achieved by diluting the feed with ultrapure water or with low-salt buffering solution.

A conditioning that aims at increasing the conductivity of the feed might be achieved by titration of the feed with an adequate amount of a salt, a combination of salts or solutions thereof, for example an adequate amount of a 5 M sodium chloride solution, until reaching the desired conductivity.

A conditioning that aims at lowering the pH of the feed might be achieved by supplementing the feed with an adequate amount of an acidic solution such as a (0.2 M to 1.0 M) hydrochloric acid solution or (1 M) acetic acid solution.

A conditioning that aims at increasing the pH of the feed might be achieved by supplementing the feed with an adequate amount of an alkaline solution such as a (0.2 M to 1.0 M) sodium hydroxide solution or a 2 M TRIS base pH 9.50 solution.

Other compounds which may be used to condition the feed prior to the multimodal chromatography step are ethanol, ethylene glycol, propylene glycol, polyethylene glycol or any other compound known in the art that further stimulates the selective binding of the protein of interest to the multimodal chromatography resin.

In some embodiments, during large scale protein purification processes, the feed is often already provided in conditions suitable for the currently disclosed method, but inadequate to be used for methods according to the prior art therefore requiring an additional conditioning step before further purification can be performed using methods of the prior art. The current disclosure thus allows such intermediate steps to be eliminated therefore reducing the number of steps during protein production processes thus increasing their effectivity and performance.

In some embodiments, in preparation for contacting the feed with the multimodal exchanger, it is common practice in the art to equilibrate the latter, in order to attain compatible conditions for loading of the feed on and binding of the protein of interest to the exchanger.

Equilibration of the exchanger may be accomplished by flowing an equilibration buffer through the exchanger to establish the appropriate pH, conductivity, concentration of salts etc. In some embodiments, the equilibration buffer may include any of a wide range of options depending on the binding requirements of a particular protein. The equilibration buffer will normally include a buffering compound to confer adequate pH control. Buffering compounds may include but are not limited to MES, HEPES, BICINE, imidazole, Tris, phosphate such as PBS, citrate, or acetate, or some mixture of the foregoing or other buffers. The concentration of a buffering compound in an equilibration buffer commonly ranges from 20 to 100 mM depending of the protein of interest. The pH of the equilibration buffer may range from about pH 4.0 to pH 9.5, more preferably 6 to 8. As mentioned above, a pH lower than 6 will generally compromise binding while a pH above 9 will result in unwanted modifications of the protein. The equilibration buffer may also comprise a salt to adjust ionic strength or conductivity of the solution as needed. Examples of suitable salts include ammonium sulfate, sodium sulfate, potassium sulfate, ammonium phosphate, sodium phosphate, potassium phosphate, potassium chloride, sodium chloride or mixtures thereof.

Elution of the protein of interest from the multimodal exchanger can be performed by the aid of an elution buffer. Both gradient elution and isocratic elution belong to the elution options. During isocratic elution, the mobile phase composition remains constant throughout the procedure. In contrast, during gradient elution the composition of the mobile phase is altered during the elution process.

When opting for a gradient elution, elution may either be done by gradually decreasing pH in the elution buffer or by gradually decreasing the salt concentration (such as NaCl) in the elution buffer or both.

In an embodiment, elution occurs by gradually and stepwise decreasing the pH of said elution buffer below 7. In a further embodiment, the pH of said elution buffer is stepwise decreased from a pH 8 to at least a pH of about 5.5 or lower. In another embodiment, elution occurs by gradually and stepwise decreasing the salt concentration of the elution buffer, to a salt concentration of below 0.5 M. In a further embodiment, said salt concentration will go from the used loading conditions such as 2 M or 1 M to below 0.5 M (while keeping the buffer concentration the same). By preference, a NaCl, (NH4)2SO4 or KPO4 solution is used. In a further embodiment, elution may be attained by both decreasing the pH and decreasing the salt concentration of the elution buffer conform the conditions as described above.

The amount of steps needed during gradient elution and the pH gradient as well as the salt gradient will depend on the nature of the protein to be purified.

Alternatively, isocratic elution is used, wherein said elution buffer will have a constant salt concentration or a constant pH. In an embodiment, said elution buffer will have a salt concentration of between 5 mM and 500 mM, more preferably 10 mM and 450 mM. By preference, NaCl, (NH4)2SO4 or KPO4 is used. In another embodiment, pH of a said elution buffer will be between about 5.5 and 7, such as 6. An example is a MES buffer of pH 6. In a further embodiment, both a combination of pH and salt concentration as described above is used for eluting the protein of interest. An example is a 50 mM MES and 350 mM NaCl buffer with a pH 6.

Elution typically occurs over about 3 to about 20 column volumes. After use, the multimodal resin may optionally be cleaned, stripped, sanitized, and stored in an appropriate agent, and optionally, re-used. Proceedings for stripping of the resin include, for example, treatment of the resin with acetic acid e.g. between 50 mM and 150 mM acetic acid.

In between loading/binding of the protein and eluting, one or more wash steps may be performed. Washing may be advantageous to re-equilibrate the column and to remove weakly bound impurities prior to elution. The wash buffer is either the same as the buffer used to equilibrate the multimodal exchanger or is conditioned to have a pH and conductivity that will result in desorption of weakly bound impurities without desorption of the target compound from the chromatography multimodal exchanger. The wash buffer may contain for example Tris, HEPES, phosphate, BICINE, MES triethanolamine, sodium chloride, ammonium phosphate, sodium sulfate and/or potassium phosphate. In some embodiments, wash conditions generally vary and must be experimentally selected for each resin/protein of interest combination.

When the buffering agent and the salt are the same chemical, the additional advantage is attained that altering of conditions such as the conductivity or salt concentration during elution can be performed without the need to re-condition the sample. Therefore, in an embodiment, the buffering chemical used is the same as the salt used for driving elution from the resin. For example, when phosphate is used as buffering agent, potassium phosphates are preferably used as salts to engage elution from the resin. This results in a faster more efficient purification process by reducing the number of steps as well as the number of chemicals/buffers required during the purification procedure, eventually also reducing the process costs.

The feed comprises at least one protein of interest such as antibodies, antibody fragments, fusion proteins, enzymes, recombinant proteins or other proteins expressed by the cells. In one embodiment, the protein of interest is an antibody, preferably a monoclonal antibody, for example a monoclonal anti-TNFα antibody. In another embodiment, the protein of interest is an antibody fragment.

In some embodiments, antibodies which can be purified according to the present method are antibodies which have an isoelectric point of 6.0 or higher, preferably 7.0 or higher, more preferably 7.5 or higher. These antibodies can be immunoglobulins of the G, the A, or the M class. The antibodies can be human, or non- human (such as rodent) or chimeric (e.g. “humanized”) antibodies, or can be subunits of the abovementioned immunoglobulins, or can be hybrid proteins consisting of an immunoglobulin part and a part derived from or identical to another (non-immunoglobin) protein. The antibody material resulting from the method as described herein generally will have a very high purity (referring to protein content) of at least 98%, preferably at least 99%, more preferably at least 99.9%, even most preferably at least 99.99%.

In some embodiments, large scale commercial processes of protein production and subsequent purification often may comprise initial purification steps, viral inactivation steps and final purification steps which are often referred to as polishing. Although required for achieving a high level of protein purity, polishing may lead to significant protein yield loss, which is to be avoided from a commercial point of view. In an embodiment, the protein purification method provided by the current disclosure is the only final purification step performed during the protein production and purification process. Due to the enhanced efficiency of the method according to the current method, one single purification or chromatography step suffices to perform the complex task of consecutive purification or chromatography steps of the prior art. The currently disclosed method thus allows to decrease the number of steps necessary during protein production and purification processes. A reduction in the number of steps further leads to a reduction in the equipment necessary for the process, the number of consumables, the time needed to perform the purification and the OPEX, all without a reduction in protein quality, purity, or yield. Accordingly, the method of the disclosure offers irresistible advantages for the industrial level production of purified proteins such as recombinant proteins, more in particular, monoclonal antibodies. Accordingly, and in another or further embodiment the method for protein purification as described herein is used as a polishing step, preferably as a polishing step during large scale protein production processes. The polishing step of the disclosure provides a method to obtain a purified protein product with a high degree of purity without compromising the yield of the production process.

In an embodiment the method for protein purification as described herein is used as a polishing step, wherein the polishing step is preceded by at least one previous step, said step could be a clarification step of a cell culture harvest or a chromatography step or both. During a protein production process, the clarification step performed on a cell culture harvest ensures removal of cell debris and other contaminants from the crude cell culture harvest. The cell culture harvest is typically obtained by culturing cells in a bioreactor. As a result of clarification, a feed comprising the protein of interest is obtained which is suited for downstream processing steps. Preferably, the current method uses a clarification step according to PCT/EP2018/058366 and US62/670,220 which content is incorporated herein by reference in its entirety. In short, the clarification step is based on the formation of floccules in the cell culture harvest, followed by efficient removal of this floccules using resulting in a feed that comprises the protein of interest.

The clarification step of the current method includes an anion exchange step. In one embodiment, the anion exchange step is a liquid anion exchange step. This liquid anion exchange step is performed, in an embodiment, by addition of electropositive compounds to the cell culture harvest. Electropositive compounds are thought to bind negatively charged components derived from host cells such as, but not limited to, nucleic acids including host cell DNA and RNA, and host-cell viruses. Accordingly, one advantage of including an anion exchange step during cell clarification is the potential contribution of the electropositive compounds to the reduction of viral components in the cell culture harvest. Electropositive compounds are provided during the clarification either in a solid phase such as bound to beads or to a depth filter, or as soluble compounds thereby performing a liquid anion exchange step.

Suitable electropositive compounds are any electropositive charged compounds such as electropositive polysaccharide, electropositive polymer, chitosan, chitosan derivatives such as deacetylated chitosan, synthetic polymers such as polydiallyl dimethylammonium chloride (pDADMAC or polyDDA), benzylated poly(allylamine) and polyethylenimine, commercially available particles like TREN (BioWorks, WorkBeads TREN, high) or cationic surfactants like hexadecyltrimethylammonium bromide (also known as CTAB) or any combination thereof. Without wishing to be bound by theory, electropositive polymers are thought to act as flocculation agents because they can simultaneously bind several negatively charged contaminants such as host cell DNA and RNA causing formation of a floc. The abovementioned electropositive compounds were found to perform extremely well (floc formation and floc size) when combined with filtration using DE according to the method of the disclosure.

In another embodiment, one or more compounds selected from the group of fatty acids having 7 to 10 carbon atoms and derivatives thereof or ureides are further added to the cell culture harvest during step (a). The different compounds of step (a) might be mixed prior to their simultaneous addition to the cell culture. This is advantageous as it reduces the number of steps necessary for obtaining a clarified cell culture. The compounds of step (a) might also be added separately, sequentially and/or alternatingly to the cell culture.

Ureides and fatty acids as described above are especially suited to (further) induce precipitation and flocculation of host cell culture associated impurities in step (a) of the method according to the disclosure. Fatty acids having 7 to 10 carbon atoms, on the one hand, are thought to exert hydrophobic interactions with hydrophobic host cell derived impurities, causing their agglomeration. Suitable fatty acids may be enanthic acid (heptanoic acid), caprylic acid (octanoic acid), pelargonic acid (nonanoic acid), capric acid (decanoic acid) or any combination thereof. The fatty acid may be added in the form of a fatty acid derivative for example a fatty acid salt, such as a sodium salt, for example sodium caprylate. Ureides, on the other hand, are thought to function as binding agents by interacting with impurities in solution, for example through hydrogen bonding. Ureides are organic compounds derived from urea and can have a cyclic or acylclic structure. Ureides include, but are not restricted to, allantoin and allantoic acid.

The compounds which are added to the cell culture will stimulate the precipitation of impurities and/or the aggregation or agglomeration of impurities, precipitates or particulates present in the harvest. Specifically, precipitated fractions contain host cell impurities, e.g. host cells, host cell proteins and host cell DNA. Flocculation of host cell impurities is achieved due to the ability of the added compounds to exert hydrophobic interactions, ionic interactions and/or hydrogen bonding with host cells related impurities present in the cell culture harvest or other mechanisms of interaction. Precipitated fractions can further include viral components present in the host cells. The cell clarification in step (a) contributes to the viral clearance of the feed in multiple ways. First, medium chain fatty acids are known to have antiviral activity and have been previously shown to cause precipitation of viral particles. Finally, electropositive compounds used in step (a) are able to bind viral particles based on electrostatic interactions with the latter. Accordingly, step (a) significantly contributes to viral clearance of the feed.

Simultaneous to the addition of the compounds described above or in a subsequent step, DE is added to the cell culture harvest and allowed to settle as a DE layer or cake on a surface, preferably a support filter having a filter surface (impermeable to DE) wherein the filter surface may not need to be a horizontal, flat or disc-shaped surface but can for example be candle-shaped. An embodiment of a specific filter is disclosed in US62/670,220 which is in its entirety incorporated by reference herein.

In short, such vessel may comprise a flexible liner wherein said filtration vessel includes at least one filter having a surface on which the dynamic filter media accumulates into a cake, said cake and at least one filter adapted, during filtration operation, to permit a filtrate including target molecules to pass therethrough and said cake, during filtration operation, adapted to prevent unwanted solid materials from passing therethrough; and a backflush source including a backflush fluid and fluidly connected to the filtration vessel via the at least one filter, said backflush source, during backflush operation, adapted to supply backflush fluid back through the at least one filter for removing the cake formed on the filter. In an embodiment, said filtration vessel includes at least one candle filter having a surface on which the dynamic filter media accumulates into a cake, said cake and at least one filter are adapted, during normal operation, to permit a filtrate including target molecules to pass therethrough and said cake, during normal operation, adapted to prevent unwanted solid materials from passing therethrough, the filtration vessel including a flexible liner for receiving the cell culture harvest solution and in fluid communication with the at least one candle filter. In an embodiment, the filtration vessel comprises a rigid or semi-rigid outer container for receiving the flexible liner. In an embodiment, an actuator is present for collapsing the flexible liner. Said flexible liner may include a drain or an agitator. In an embodiment, at least one candle filter is suspended within the flexible liner.

Such DE cake will contain a structure comprising a plurality of channels or paths. Upon filtration of the cell culture harvest comprising the floccules through the DE layer or cake, the large matter such as cells, cell debris, and other large non-target compounds of the solution obtained) are retained by the DE cake structure, whereas the target proteins, having a smaller size, flow through the channels of the DE cake structure. To facilitate such flow, a pump or other pressure dispense aid or other fluid driving mechanism is used as further described in embodiments below.

The use of DE to facilitate filtration overcomes the limitations imposed by the physical changes of the cell culture after flocculation which tend to render routine clarification complex. For instance, in case of depth filtration, a very significant surface of depth filters is needed to clarify the first solution comprising the formed precipitates. Use of DE allows achieving significant operational advantages including shorter processing time, less process steps, less process materials, less equipment and solutions, without compromising the quality of the cell clarification step. Finally, filtration of the flocculated cell culture harvest results in a feed comprising the protein of interest.

A chromatography step preceding the polishing step according to the abovementioned embodiment of the method of the disclosure can refer to, for example, a liquid ion exchange chromatography performed as part of the clarification process. In a further embodiment the chromatography step preceding the polishing step can refer to a protein capture step based on an ion exchange chromatography step or an affinity chromatography step performed on the clarified cell culture harvest prior to the polishing step. In another or further embodiment, the feed for multimodal chromatography polishing according to the disclosure is a flow-through fraction of a chromatography step or a fraction derived thereof. Removal of key impurities in steps preceding the polishing step according to the current disclosure allows for an improved performance of the latter.

During a protein process, a feed comprising a protein of interest and viruses will typically be subjected to a virus inactivation step or process. After virus inactivation, the virus or viral particles are still present in the feed and need to be removed in an efficient manner, thereby ensuring high protein yield and purity. Viral inactivation may have been achieved in a step preceding the polishing step according to the current disclosure by use of a low pH, thereby inactivating the viruses in the feed, accordingly the feed may comprise inactivated viruses or viral particles. By performing the current method for protein purification after viral inactivation, a very pure protein with a high yield is obtained.

Chromatography steps according to the disclosed method can be performed in batch mode. Alternatively, said chromatography steps are performed in continuous mode. When performed in batch mode, said chromatography step is repeated over one column (single-column batch) or multiple columns (parallel batch) until all the feed has been loaded and subsequently eluted. The eluates or pooled eluates, respectively, are optionally pooled before proceeding to the next purification step. Alternatively, the chromatography steps can be performed as a continuous process where each step of the purification method is performed simultaneously and a single column or multiple columns are loaded and eluted continuously. While the classical batch-operation sequence does not require specific adaptations of the equipment and often results in a protein of high purity, the current method is well suited to be performed in a parallel column continuous mode process. A continuous mode offers the additional advantages that higher productivity can be achieved as the efficiency of the protein purification process is increased. In addition, the continuous mode helps reduce the protein purification costs by reducing the amount of consumables needed for a larger purification scale. Continuous mode chromatography, for example, allows to reduce chromatography column size without sacrificing the productivity of the purification step.

It is supposed that the present invention is not restricted to any form of realization described previously and that some modifications can be added without reappraisal of the appended claims.

### DETAILED DESCRIPTION OF FIGURES

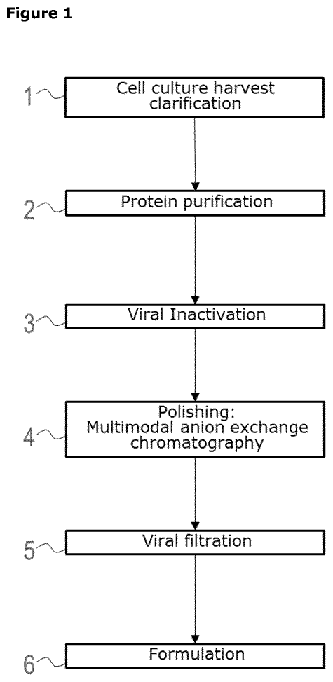

FIG. 1 is a flow chart depicting an overview of a large-scale commercial process of protein production and purification.

Large scale commercial processes of protein production and subsequent purification often comprise initial purification steps, viral inactivation steps and final purification steps which are often referred to as polishing. Although required for achieving a high level of protein purity, polishing may lead to significant protein yield loss, which is to be avoided from a commercial point of view.

The clarification step 1 is performed on a cell culture harvest and ensures removal of cell debris and other contaminants from the crude cell culture harvest. The cell culture harvest is typically obtained by culturing cells in a bioreactor. As a result of clarification, a feed comprising the protein of interest is obtained which is suited for downstream processing steps.

A first protein purification 2 preceding the polishing step according to an embodiment of the method can comprise a protein capture step based on an ion exchange chromatography step or an affinity chromatography step performed on the clarified cell culture harvest.

During a protein process, a feed comprising a protein of interest and viruses will typically be subjected to a virus inactivation step 3. Viral inactivation may be achieved in a step preceding the polishing step according to the current disclosure by use of a low pH, thereby inactivating the viruses in the feed, accordingly the feed may still comprise inactivated viruses or viral particles.

The methods of the prior art combine several polishing steps and an intermediate ultrafiltration or depth filtration step to achieve a high degree of purity of the purified protein of interest. When the current method for the purification of a protein is used as the polishing step 4, the multiple steps of the prior art methods are reduced to a single step, based on the use of multimodal ion exchange chromatography step. In FIG. 1, anion exchange is shown as an example, but the skilled in the art will understand that cation exchange equally belongs to the options.

Use of multimodal ion exchange results in the reduction of purification process time, space and consumables without compromising the degree of protein purity at the end of the purification process. The polishing step 4 leads to the purified protein of interest.

Optionally, the purification process includes an additional step of viral filtration 5 and a formulation step 6.

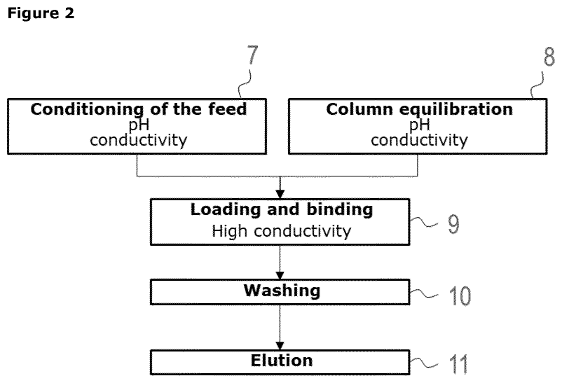

FIG. 2 is a flow chart depicting an overview of the method for purifying a protein according to an embodiment of the disclosure.

The depicted embodiment of the disclosed method for the purification of a protein of interest employs a multimodal anion exchange chromatography step. Loading of the feed and binding 9 of the protein of interest onto the multimodal chromatography resin according to this embodiment occurs at a pH of about 7 to 9 and a high conductivity of at least 75 mS/cm. The high conductivity conditions result in a shift towards the hydrophobic properties of the multimodal exchanger, whereby interaction of the protein of interest with the multimodal ion exchanger is based on the hydrophobic properties of said protein and the hydrophobic moiety of the multimodal ion exchange ligand. This shift allows for successful modulation of the separation properties of the multimode chromatography resin, e.g. selectivity towards the monomeric form of a protein, e.g. antibody. In addition, the shift in chromatography also allows to selectively separate antibody charge variants from one another. Simultaneously, all major attributes of anion exchange chromatography such as reduction of host cell proteins and DNA, as well as reduction in viral particles are preserved as well. Hence, the method allows obtaining a highly pure protein sample, of a high quality.

The conditions of the feed comprising the protein of interest as well as the chromatography resin are adjusted by conditioning 7 and equilibration 8 towards the conditions as described above, prior to performing the multimodal chromatography step, or may already comply to the conditions as described. Conditioning 7 of the feed to a high conductivity can be performed by supplementing the feed with an adequate amount of salt or a combination of salts.

After loading and binding 9 of the protein of interest to the multimodal ion exchange resin, the resin is washed 10 twice with 3 to 20 times the column volume. This allows to remove unbound and weakly bound contaminants from the column.

The purified protein is then eluted from the multimodal exchange chromatography resin using either isocratic or gradient elution. In the current embodiment, isocratic elution is performed with an elution buffer, which has a salt concentration of between 250 mM and 500 mM and a pH of between 5.5 and 6.6.

### EXAMPLES

The following examples are meant to further clarify the disclosure but are not to be seen as a limitation of the latter.

### Example 1

### Purification of an Antibody

A protein solution containing an antibody of interest was passed through a multimodal chromatography matrix, Capto Adhere Impres, GE Lifesciences, in bind-elute mode using a AKTA 150 chromatography system. The column was equilibrated with a 50 mM HEPES buffer, 2 M NaCl, pH 7.0. Following binding to the column matrix at 30 g/I, the column was washed with a 50 mM HEPES buffer, 1.5 M NaCl, pH 7.0. The protein of interest was eluted from the column by isocratic elution with a 50 mM HEPES buffer, 0.35 M NaCl, pH 7.0. More than 98% purity of the antibody of interest is achieved after this column step, as assessed by SEC-HPLC.

### Example 2

About 10mL of affinity purified Adalimumab antibody having a concentration of 7mg/mL originating from 10L cell culture was used for this example.

The pH of the solution after neutralization was pH 7.0 where the neutralization was done using 2M Tris-base solution having a pH of 9.4. By using this solution, the pH of the affinity eluted protein was raised from pH 3.5 to pH 7.0. After neutralization the conductivity of the affinity chromatography eluted major fraction was 5.4 mS/cm. The chromatography eluted major fraction was reconditioned using concentrated 5M NaCl solution to conductivity of about 92 mS/cm. This salt concentration matched the binding buffer conductivity of the same. The pH of the solution was also adjusted using 150pL of 5M NaOH to pH 7.0.

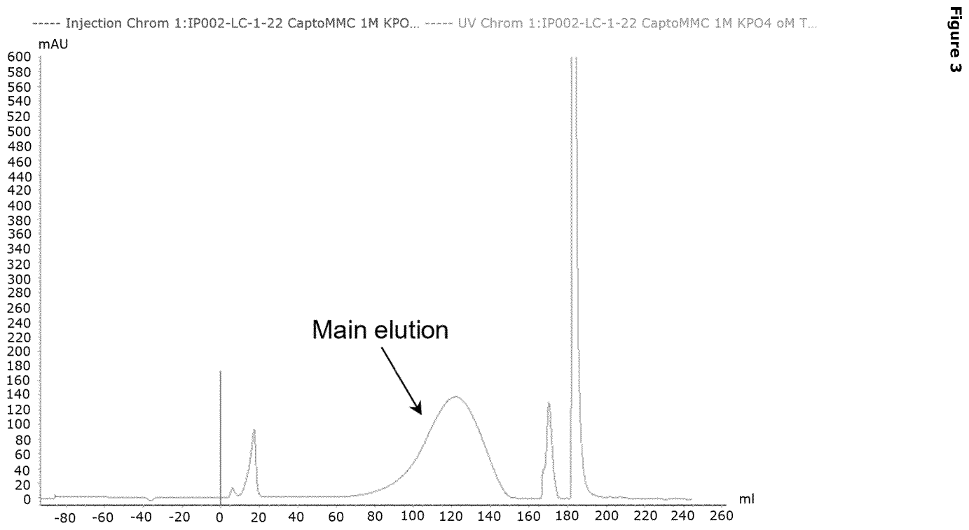

After the reconditioning step the protein was loaded onto a mixed mode cation exchanger Capto MMC HiScreen (0.8×10cm) column, with a bed volume of 4.7mL fitted to AKTA 150 chromatography system (GE Lifesciences), at 200cm/hr. Prior to loading, the column was equilibrated with a 1M KPO4 buffer, pH 7.0. The loading was done at 14 mg/mL of matrix and 3 min residence time, the column was washed with the same buffer (first wash). Following the first wash step, the protein was eluted from the column with a 10 mM KPO4 buffer, pH 7.0. The chromatogram confirmed excellent yield of the protein of interest (see FIG. 3). Additional examples of embodiments according to the current disclosure are provided in Table 1.

TABLE 1
Multimodal chromatography conditions according
to embodiments of the disclosed method

| | Conditions A | Conditions B | Conditions C |
|---|---|---|---|
| Equilibration | High conductivity conditions: | | High conductivity conditions: |
| and binding | up to 2M NaCl or up to 1M KₓPO₄ | | up to 2M NaCl or |
| conditions | High pH: | | up to 1M KₓPC₄ |
| | up to pH 8.5 | | Neutral pH |
| Wash | Wash 1 - same as equilibration | | |
| conditions | Wash 2 | Wash 2 | Wash 2 |
| | Slightly lower | Slightly lower | Slightly lower |
| | conductivity | conductivity | conductivity |
| | Unchanged pH | Slightly lower pH | Neutral pH |
| Elution | Gradient elution: | Gradient elution: | Isocratic elution: |
| Conditions | Descending | Descending | Significantly lower |
| | concentration of salt | concentration of salt | concentration of salt at |
| | at constant pH | and descending pH | constant pH |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for purifying a protein of interest, wherein said protein is present in a feed, and wherein said method comprises a multimodal chromatography step, wherein said feed is contacted with a multimodal ion exchanger comprising a ligand with a hydrophobic moiety and a charged moiety and wherein binding of said protein of interest to said exchanger occurs under high conductivity conditions.

2. The method according to claim 1, wherein said feed is supplemented with an adequate amount of salt or a combination of salts prior to said multimodal chromatography step.

3. The method according to claim 1, wherein said feed is supplemented with an adequate amount of ammonium sulfate, sodium sulfate, potassium sulfate, ammonium phosphate, sodium phosphate, potassium phosphate, potassium chloride, sodium chloride or a mixture thereof prior to said multimodal chromatography step.

4. The method according to claim 1, wherein the salt concentration of said feed during binding is between 0.5 M and 3 M.

5. The method according to claim 1, wherein said salt concentration of said feed during binding is between 1 M and 2 M.

6. The method according to claim 1, wherein said charged moiety is positively or negatively charged.

7. The method according to claim 1, wherein said multimodal exchanger has both positively and negatively charged moieties.

8. The method according to any of the previous claim 1, wherein said multimodal exchanger comprise additional moieties, allowing additional interaction functionalities other than ion exchange of said column, such as hydrophobic-interaction-enabling moieties or hydrogen-bonding-enabling moieties.

9. The method according to claim 1 wherein said feed is supplemented with an adequate amount of an acidic solution or with an adequate amount of an alkaline solution prior to multimodal chromatography step.

10. The method according to claim 1, wherein said binding occurs at a pH of about 7 to 9.

11. The method according to claim 1, wherein the multimodal chromatography step is used as a polishing step.

12. The method according to claim 9, wherein said multimodal chromatography step is the sole polishing step.

13. The method according to claim 9, wherein said polishing step is preceded by a clarification step of a cell culture harvest and a chromatography step.

14. The method according to claim 1, wherein said protein is eluted from said multimodal exchanger by gradient elution, by gradually decreasing the pH of an elution buffer below 7 and/or by gradually decreasing the salt concentration in an elution buffer below 0.5 M.

15. The method according to claim 1, wherein said protein is eluted from said multimodal exchanger by isocratic elution with an elution buffer, wherein said elution buffer has a salt concentration of between 10 mM and 500 mM and/or a pH of between 5.5 and 7.

16. The method according to claim 1, wherein said feed comprises inactivated viruses.

17. The method according to claim 1, wherein the feed for multimodal chromatography is a flow-through fraction of a chromatography step or a fraction derived thereof.

18. The method according to claim 1, wherein said protein is an antibody.

19. The method according to claim 1, wherein said method is performed in batch mode or continuous chromatography mode.

20. A kit comprising:
a multimodal chromatography resin comprising a ligand with a hydrophobic moiety and a charged moiety; and
a buffer with a salt concentration of between 0.5 and 3 M and/or a conductivity of above 75 mS/cm.

21. A multimodal ion exchanger comprising a ligand with a hydrophobic moiety and a charged moiety; and a protein bound to said hydrophobic moiety.

22. The multimodal ion exchanger according to claim 21, wherein said protein prior to loading is present in a buffer with a salt concentration of between 0.5 and 3 M and/or a conductivity of above 75 mS/cm.

23. The multimodal ion exchanger according to claim 21 comprising a buffer with a salt concentration of between 0.5 and 3 M and/or conductivity of above 75 mS/cm.

24. The multimodal ion exchanger according to claim 21 comprising a buffer at a pH of about 7 to 9.

25. The multimodal ion exchanger according to claim 21, wherein said multimodal exchanger comprise additional moieties, allowing additional interaction functionalities other than ion exchange of said column, such as hydrophobic-interaction-enabling moieties or hydrogen-bonding-enabling moieties.

26. A protein purification system comprising a multimodal ion exchanger comprising a ligand with a hydrophobic moiety and a charged moiety, the system further comprising a protein feed having a pH between about 7 and about 9, and a salt concentration between about 0.5 M and about 3 M.

27. The protein purification system of claim 26, wherein the hydrophobic moiety and the charged moiety are on separate ligands.
